(19) United States
(12) Patent Application Publication
(10) Pub. No.: US 2012/0316392 A1
(43) Pub. Date: Dec. 13, 2012

(54) SPHERICAL CAPSULE VIDEO ENDOSCOPY

(76) Inventor: Séraphin Nicaise Itoua, Lausanne (CH)

(21) Appl. No.: 13/576,504

(22) PCT Filed: Feb. 1, 2011
(86) PCT No.: PCT/IB11/50432

§ 371 Date: Aug. 30, 2012

(30) Foreign Application Priority Data
Feb. 1, 2010 (EP) 10152293.6

(51) Int. Cl. A61B 1/04; A61B 1/06

(52) U.S. Cl. 600/111; 600/109

(57) ABSTRACT

An imaging device, for example a endoscopic capsule, comprising a core base (1) having a plurality of faces, said faces supporting at least one an imaging device with an illumination means, said device further comprising electronic means and being contained in a spherical envelope (8).

### FIELD OF THE INVENTION

The present invention concerns a spherical video capsule that can be used, for example, for endoscopic applications.

In addition to medical indications such as the endoscopic applications mentioned above, the proposed capsule can also be used in other fields, to inspect canalisations in factories. For example, in milk industry and other drink industries, the quality of soldering and the state of canalisations need to be explored. In such applications the capsule of the present invention will preferably contain memory means to save images, since wireless communications may be difficult in some applications. The capsule principle is the same, but the size may be adapted to each application.

Other applications are of course possible in addition to the one mentioned in the present patent application.

### BACKGROUND OF THE INVENTION

Diseases of the digestive tract are numerous. For many years, the diagnosis of these diseases was mainly done via endoscopy: a cable with a camera. The small intestine has long been a difficult organ to explore in terms of endoscopy. Only the duodenum, the proximal jejunum and terminal ileum are accessible by conventional endoscopy. The advent of enteroscopy and capsule endoscopy has completely changed the management of pathologies related to the small intestine.

Other diseases where an endoscopic video camera is potentially useful include inter alia:
The search for locations of the intestinal graft reactions against the host
The search for localization of gastrointestinal general illness, such as amylose digestive disease, Waldmann disease and hypogammaglobulitémie.

The limits of the examination of the small intestine by the current video capsule are related to the quality of the images that are at the level provided by the endoscopes in the 1980s but actually depends on the contents of the small intestine at the time of examination, hence the interest of a pre-operative preparation.

The big difficulties with the use of the present capsules are:
inability to precisely locate lesions:
the system proposed by the manufacturers is too vague:
only the appearance of small bowel mucosa (height of the folds, thickness . . . ), the transit time from the pylorus and from the ileocecal valve can roughly locate the lesion.

After saving images on a computer disc, a workstation uses a program for automatically detecting the presence of potentially bleeding lesions. Unfortunately the sensitivity is low (below 50%) and therefore the images must be viewed in their entirety.

The cost of an actual endoscopic capsule is about 650 Euros and currently, three capsules are available in Europe (all incorporated by reference in the present application as to the means and principles of such devices): the one commercialized by Given Imaging (M2A Capsule) since 2002, the one from Olympus since 2005, and the one from Intromed.

In the field of video capsule endoscopy, technical progress expected by the medical community is:
Capsules controlled progression
Recovery systems retained capsules
Impermeable capsules
Increase the recording time and number of images per minute.
A better precision in the lesions localization

The chronic gastrointestinal bleeding of undetermined origin (hidden or externalized) is a useful indication for using a video-capsule.

During the assessment of chronic gastrointestinal bleeding; abnormalities most frequently pointed out are the following in decreasing order of frequency: arterial venous malformation, ulcerations secondary to NSAIDs, ulcerative lesions of Crohn's disease, ulcerated tumors and Dieulafoy ulcers.

Multiple studies have demonstrated the effectiveness of an endoscopic capsule in diagnosing the source of chronic gastrointestinal bleeding with a diagnostic yield of 55-80%. This diagnostic yield is consistently superior to enteroscopy, atenteroscopy, preoperative or double balloon enteroscopy.

All other conditions affecting the small intestine can theoretically be investigated by an endoscopic video camera. But the real impact of the endoscopic video camera in the management of these diseases remains to be determined and is the subject of many studies. The video capsule enables to highlight the mucosal surface, to highlight colour changes without necessarily be able to determine a diagnosis or even define the pathological nature of the image encountered.

The advantages of an endoscopic video camera are the following (non exhaustive list):
It is a non-invasive act, requiring no sedation, no insufflation of the intestine.
It can explore the entire small intestine.
It does not require disinfection since the capsule may be disposable. Morbidity related to the examination is reduced.
The technique to use such a capsule is easily learned.

The disadvantages of the endoscopic video camera are the following:
The review does not allow biopsies that are often useful in interpreting images and does not achieve a therapeutic gesture. It is not 100% reliable: lesions may be missed by EVC.
There are counter-indications.
The playing time is long and expertise of gradual recognition of lesions. Its cost is high.

The known Given Imaging capsule consists of a cylinder 11 mm in diameter and 26 mm long and weighs 3.7 g. It is made of a biocompatible material resistant to the action of digestive enzymes. It consists of a dome and optical lenses for a field of vision of 140 degrees.

In this capsule, the intestine is illuminated through this dome of light emitting diodes (LEDs) and the acquired images are focused on a 65,000-pixel camera (CMOS). This image captures, transforms into an electronic signal and via a transmitter (located at the other end of the capsule) transfers it to 8 sensors on the patient's abdomen. Images captured at 2 per second will be recorded in a case brought by the patient in his belt (like a holster). The duration of image transmission is a function of battery capacity, i.e. roughly eight hours. The capsule is clearly disposable and is removed by a natural process in 24 to 48 hours. The images are received can be stored and processed on a microcomputer.

In addition, other capsules already exist:
The esophageal capsule has the same size as the classic dish but captures images at each end at a frequency of 14 frames per second. The patient should not eat or drink for 3 hours before the intervention. This capsule does not allow to diagnose the biopsies.
The colon capsule is in full development. It is slightly longer than the standard capsule (32 mm×11 mm), captures images with 2 ends at a frequency of 4 frames per second. The capsule is swallowed up in active mode, then after a couple of minutes is turned off, to spontaneously turn on again after 2 hours, in order to save power and allow maximum viewing of the colon. Just like any other examination, colonic optimal preparation is required. The studies cited confirm the feasibility of the technique and pave the way towards the study of pathologies, particularly in a context of screening for possible colonic polyps.

A capsule is known from document JP 2006068109, incorporated by reference in the present application. In this prior art, the capsule comprises four CCD cameras arranged at vertices of a regular tetrahedron and has a position sensor made of a sphere connected with spring to detect an abnormality in the position of the capsule according to the expansion or contraction stress acting on the springs.

### SUMMARY OF THE INVENTION

An aim of the present invention is to improve the known devices and methods.

More specifically, it is an aim of the present invention to propose a spherical capsule equipped with multiple image sensors which are able to take pictures in all directions.

Another aim of the present invention is to propose a spherical capsule that may be oriented when used, for example in the small intestine of a patient, or in another suitable application, in order to improve the images taken of the environment of the capsule.

Because of the full 360° spherical view, the proposed capsule does not really need to be oriented. Its main required driving feature is to be slowed down. The acceleration feature may be provided by natural behaviour of the digestive tract. However, these capabilities do not exclude introduction of custom driving features.

The device, apparatus and method according to the present invention are defined in the appended claims.

### BRIEF DESCRIPTION OF THE DRAWINGS

The present invention will be better understood by reference to the following detailed description and to the figures that show

FIGS. 1 to 3 illustrate the principle of the capsule according to the present invention;

FIGS. 4 and 5 illustrate the principle of means for moving the capsule of the invention while being used;

FIGS. 6 to 8 illustrate embodiments of imaging means used in the capsule according to the invention;

FIGS. 9 to 11 illustrate the use of the capsule of the present invention in an endoscopic application;

FIGS. 12 and 13 illustrate geometrical transformations (translation and rotation of a point);

FIG. 14 illustrates a reconstructed 3D object (for example a part of the digestive tract);

FIGS. 15 and 16 illustrate images taken by the capsule according to the invention;

FIGS. 17 and 18 illustrate an embodiment of the present invention;

FIG. 19 illustrates another embodiment of the present invention;

FIGS. 20 and 21 illustrate pairs of images taken by the capsule of the invention.

### DETAILED DESCRIPTION OF THE INVENTION

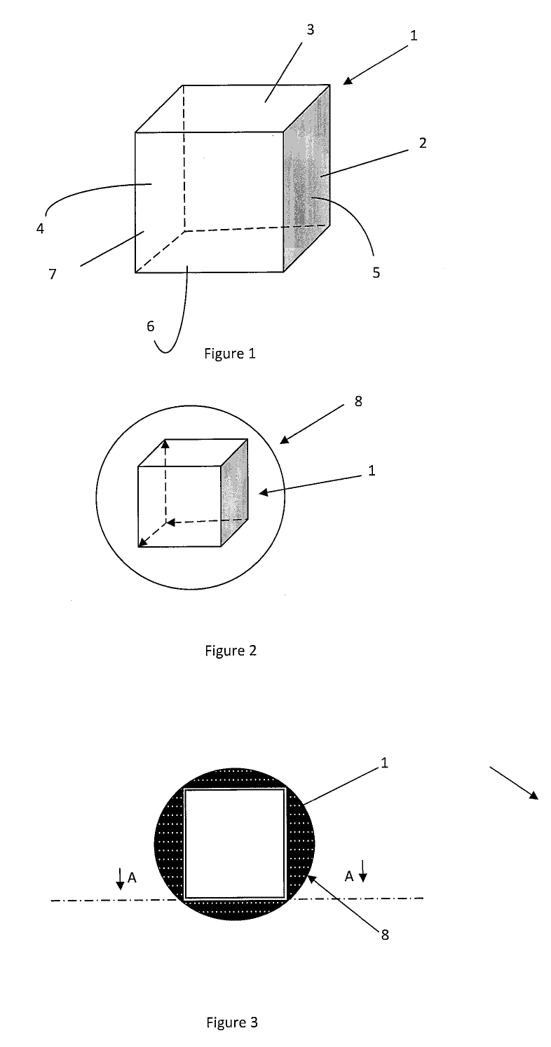

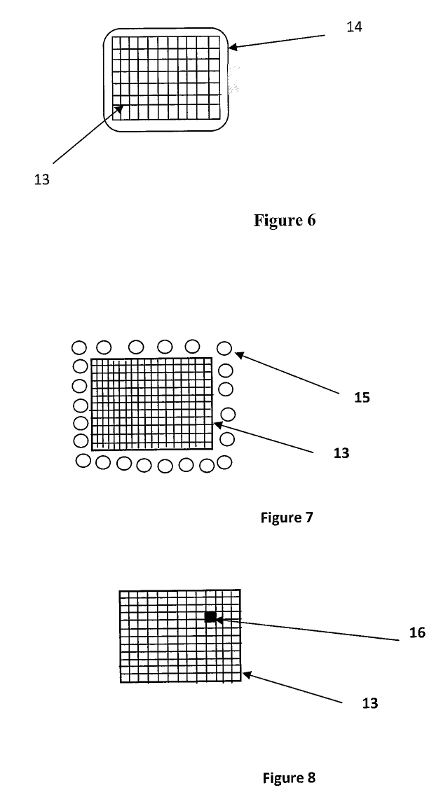

FIG. 1 illustrates a core base 1 which is cube-shaped. Each of the six faces 2-7 is equipped with an image sensor, which will be described in more detail with reference to FIGS. 6 to 8 below. Using this cube, we have therefore at least six image sensors (for example cameras).

In a variant using image sensors having a very large viewing angle, their number could be reduced to four, for example, still reaching the desired result as described in the following.

However, it is to be noted that the number of image sensors is not limited to six. Indeed, by using a non-cubic shape having more than six faces it is possible to install more than six sensors on the core base 1.

The extreme case of this extension is to consider the situation where the central core base 1 is no longer a cube anymore but a sphere where each point becomes a pixel and is therefore an image sensor. In this case, there will be a multitude (i.e. a lot more than six) of image sensors.

Whatever the shape of the core base 1 carrying the image sensors, this support is embedded in a glass sphere 8 or a sphere 8 made of another equivalent material suitable for the intended use. This means that the final object is substantially spherical in shape. Indeed, the video capsules endoscopy proposed so far in the prior art all have an oval shape and they sometimes suffer blockage during their motion because of their shape. By adopting a substantially spherical shape, the capsule according to the present invention will find it easier to roll and then to circulate when being used. Therefore, it should experience less blocking and other problem arising with conventional capsules that have an oval shape.

As indicated above, the capsule has a substantially spherical shape, thus the core base 1 is embedded into a transparent spherical casing 8 (see FIGS. 2 and 3) which may be made in any suitable material for the intended application as mentioned previously.

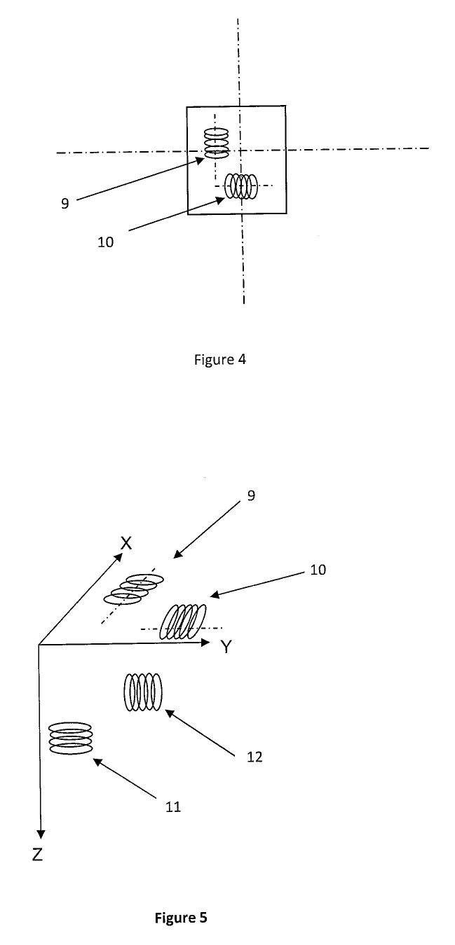

FIG. 4 illustrates a detail of elements of the capsule according to the present invention, taken along the line A-A of FIG. 3. This figure shows two inductors 9, 10 (for example coils) which are used to orient the capsule. More specifically, the capsule 1 comprises two inductors that are embedded on the internal side of a printed board which form a face 2-6 of the capsule 1. However, there is no need of having two inductors for each printed board, i.e. each face of the capsule supporting an imaging device: the inductors 9, 10 might be implemented in three PCB (printed circuit board), among six (when the core base comprises six faces). The choice of PCB containing coils is done so that there is one pair of them in each axis-direction, i.e. in each plane: (X,Y), (X,Z), and (Y,Z), see FIG. 5, pair of coils 9, 10, 11 and 12.

The purpose of these coils 9-12 is the implementation of driving capabilities of the capsule by an external magnetic field generated by known means. Through these coils and their disposition, the capsule may be oriented and its speed may be reduced to improve the visible surface of digestive tract and take more images of the environment if necessary.

FIG. 6 illustrates a matrix of image sensors 13 and possible antenna 14 which is optional.

The antenna 14 can be installed around each image sensor array 13, and this is not the only place possible. The antenna may be used to transmit data (i.e. image data) from the capsule to an outside device for subsequent treatment. Typically, this would include real time imaging on a screen as well as data treatment for example to improve the received data.

An antenna may also be used to transfer energy from the outside to the capsule to the means contained in the capsule for example.

The sensor array is used as imaging means to take pictures of the environment of the capsule. Typically, one may use the following as sensor array: CMOS or CCD or other equivalent devices. However, due to the fact that the capsule is mainly used in dark places, it is necessary to add illumination capabilities in order to illuminate the zone being captured by the imaging device.

For illumination purposes, there are several embodiments as described in the following.

For example, in the embodiment of FIG. 7, LEDs (Light Emitting Diodes) 15 are placed outside the sensors array 13. These LED are discrete devices, i.e. inserted on the board (face) or integrated on the chip, but at the border. The LED number count in FIG. 7 is only a non-limited example.

Alternatively, each pixel of the imaging device 13 can be equipped with a LED 16 to illuminate the area to shoot and circuitry of the pixel. This embodiment is illustrated in FIG. 8.

Of course, both embodiments may be combined together in accordance with circumstances and improve the device according to the invention thus using at the same the two configurations. A given capsule could possess both configurations and each may be used according to circumstances (if one is better than the other).

Electronic devices (electronic monitoring, transmission, power, etc. . . . ) are housed in the central core. Typical elements included are at least a microcontroller, memories (RAM and ROM), at least one ASIC (Application Specific integrated Circuit), a battery or energy source, receiver/transmitter, amplifiers, modulator, demodulator, filters, voltage regulators, rectifiers. These components are mainly integrated in monolithic chips and some of them can be discrete, i.e. out of the chip. This can be a single ASIC driving all sensors, and that includes all listed analog, digital and mixed functions. This ASIC may also be spread full or partly in all sensors. One can imagine image sensors (CMOS or CCD) made as a 3D-chip. In fact, instead of spreading a layout of a chip over a surface, as in conventional design, 3D-IC (Three Dimensional Integrated Circuit) design allows superposition of blocks in a multilayers organisation on the same surface. This approach allows chips size optimization, and would be suitable for the described capsule, since it would reduce the count of components inside the cubic box.

When the 3D-IC design approach is chosen, the top bloc is of course the image sensor. Other functions, as ASICs, are placed under the sensors.

The transponder antenna that brings energy is driven by the ASIC. That is also the case of coils that slow down the device. All the actions are under the control of the ASIC.

The sensors and other electronic devices (ASIC, etc.) are of course connected together through wiring or wireless connectivity. This allows the capsule to be considered as a single system/device.

The embodiment proposed for the imaging devices makes it possible to cover the entire sphere: that is to say all angles around the sphere. So there is no blind spot. Moreover, the image resolution may be contained in a wide range: up to high definition (HD).

The shots taken by the capsule can be small or up to 30 frames per second or more. This allows for the real video.

With a good resolution and a host of images of real video, it would probably extend the diagnostic of diseases hitherto unexplored by other capsules. For example, passing simultaneously the images from the six image sensors, it is possible on a single screen to track the exploration of the digestive tract without missing a thing. Software image processing will be proposed for this purpose.

Increasing the frame rate has the effect to increase the amount of information transmitted, but this opens the way for the kind of video compression MPEG. Indeed, a rate of 4 frames per second example is a limitation in the quality and compression.

The main weakness of current capsules is the difficulty of determining its effective position. Indeed, when a picture or image shows a place of interest, for example a tumor, the known capsules are not able to indicate where the image was taken. However, surgeons want to know this information in order to go straight to the point and act on a specific place where the place of interest has been identified.

To solve this positioning problem, some capsules are based on multiple sensors inserted under the skin of the patient in different places. This technique is similar to that of GPS (Global Positioning System). Such a technique cannot give satisfactory results because a signal sent by the capsule does not necessarily directly reach the sensor due to multipath and because the spreading does not occur in free space. As a consequence, practitioners do not trust this positioning technique which is not precise.

The proposed solution in the present invention is to use a positioning system with a local reference. More specifically, the proposed solution relies on the fact that several image sensors are fixed on the same physical media: cube, or other form.

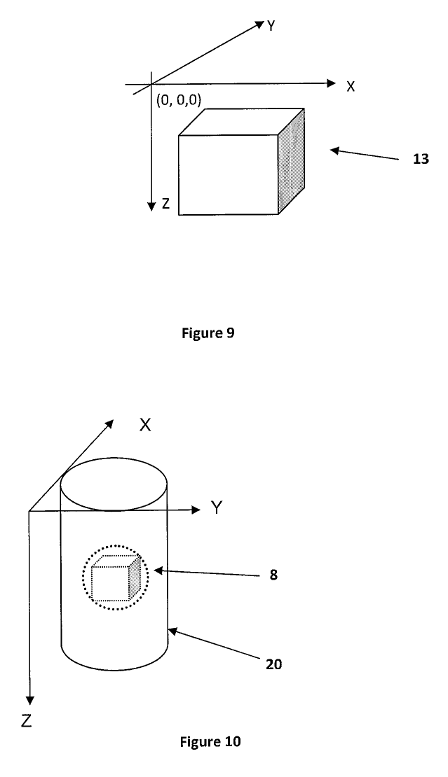

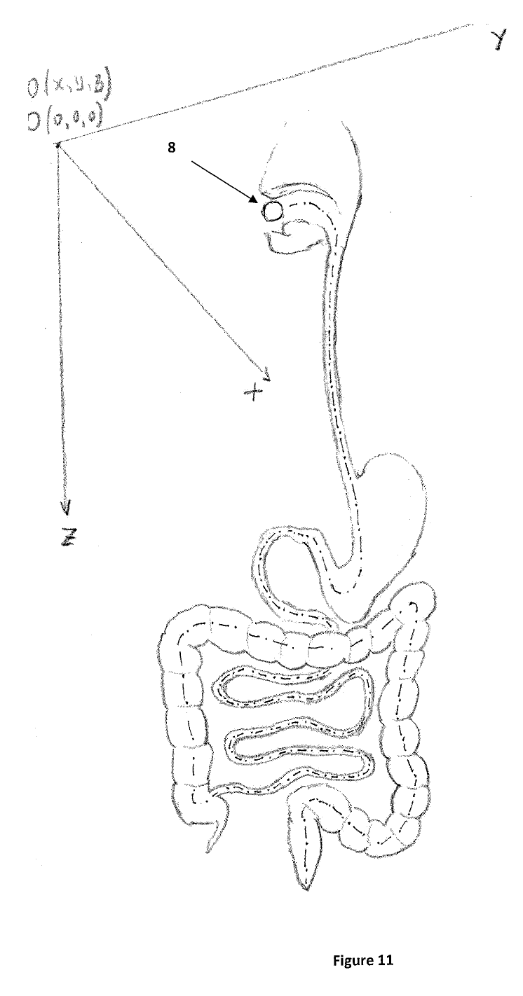

To explain the present solution, an example is described in relation to the digestive tract and the digestive tract is considered to be placed in a three-dimensional X, Y, Z coordinate system, see FIGS. 9, 10 and 11.

The origin of the coordinate system is point O, with coordinates (0,0,0). Once the capsule is placed in the mouth by the patient (as an exemplary origin of the coordinates), the capsule is activated by an appropriate means. This activation can be done for example by a radio signal received by the antenna 14 (see FIG. 6). Upon activation, the first images are taken by all cameras simultaneously. Considering the trajectory of the capsule, the place where the first images are taken can be considered the starting point of the trajectory and observed O′. Its coordinates are not all zero from the point O.

This point of the first shots will mark the landmark. Activating this shooting in the patient's body prevents that the coordinate system is located outside the gastrointestinal tract: therefore, we can talk about local or relative positioning.

From that moment, every movement of the capsule is identified relatively to the initial point O′(x′, y′, z′). This point may be associated with the center of the sphere of the capsule.

Every movement of the capsule allows new images on each face of the cube 1 to be taken where imaging devices 13 are present.

Each series of images is associated with a position of the capsule, i.e. a point whose coordinates are known because they can be detected by analyzing two consecutive sets of images.

The capsule being spherical, the imaging devices are equidistant. From these shots, it is thus possible to:
Reconstruct the digestive tract in 3 dimensions as an object volume to facilitate understanding by doctors or surgeons;

By analyzing the images taken by each camera, to identify the movement in the sphere in a 3D space, and therefore its trajectory.
Having visibility in all directions of the sphere, any movement of the capsule (translation, rotation, translation with rotation) can be calculated.

The print of such a path does not rely on references outside the capsule, but only its own images. One can therefore speak of an intrinsic position with no reference to time. This trajectory can be made of elementary operations on images: move, rotate, etc. . . . . These are simple techniques well known in image analysis and processing.

Having reconstructed the trajectory of the capsule, this trajectory can then be regarded as the mainstay of the type the patient's digestive considered. Then, from images taken by each camera, it is possible to make a 3D reconstruction similar to that used in tomography.

If the shots are close together (eg, 30 frames per second), we can be certain of detecting all movements capsule: rotation, translation, etc. Knowing the positions of the cameras against each other, image analysis can determine the distance travelled by the capsule, the rotation carried out and the X, Y, Z of each shot. Initially, the X, Y, Z can be expressed in pixels. They can find their equivalent in the metric system because the pixel size is known and the image analysis technique such as mathematical morphology, remote sensing contribute in this direction.

What is important here is the initial starting point. The coding of the coordinates can even be done in different ways: by referring to the initial point (0, 0, 0) or in relative or in a row. Note that the trajectory of the capsule can be calculated in real time as well as a posteriori, i.e. after saving the images. It is the same for the choice of benchmark O′, which can be arbitrary, but in any case it must be located inside the patient's body.

### Mathematical Description of the Problem for Following the Trajectory of the Capsule.

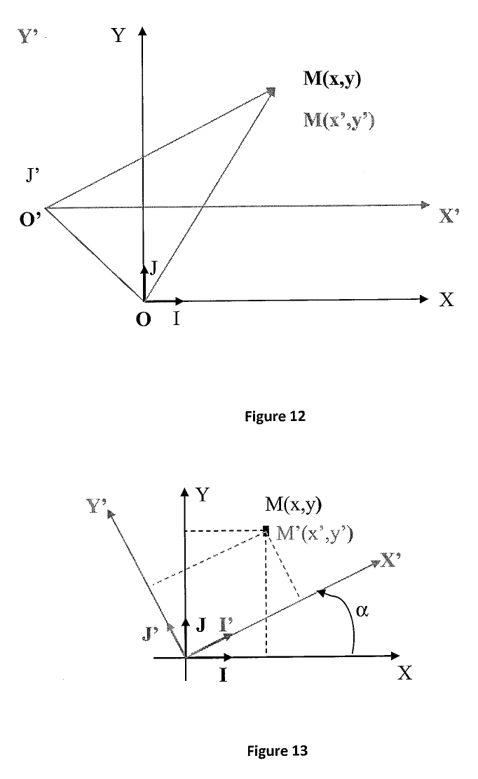

Coordinate transformation on a parallel translation on a plane (see FIG. 12 illustrating the translation on a plane and FIG. 13 illustrating the rotation on a plane) or a system of X, Y coordinates with the origin point O. Consider a point M located on the plane and having coordinates x, y. Consider that movement concerns only the origin of the marker (FIG. 12). Let O′(a, b), the new origin and X′ and Y′ axes of the new landmark. We can then write: OO′=OM+O′M.

Therefore, x=x′+a, y=y′+a. (1)

Thus, we obtain the formulas for transforming the old coordinates of the point M in new coordinates. From the expression (1), we obtain:

$$\{ \begin{matrix}x = {x}^{′} - a \\ y = {y}^{′} - b\end{matrix} \qquad ( 2 )$$

Rotation of Axis with Maintaining of the Origin (FIG. 13)

In an orthogonal coordinate system, we have:

$${I}^{′} = \{ cos α , cos ( \frac{π}{2} - α ) \} = \{ cos α , sin α \}$$

$${J}^{′} = \{ cos ( α + \frac{π}{2} , cos α ) \} = ( - sin α , cos α )$$

Let M a point of the plane and (x, y) its old coordinates, and (x′, y′) the new coordinates.

In this case,

$$\begin{matrix}x = {x}^{′} cos α - {y}^{′} sin α \\ y = {x}^{′} sin α - {y}^{′} cos α\end{matrix} \} \qquad ( 3 )$$

that means:

$$\begin{matrix}\vec{OM} = x \vec{I} + y \vec{J} \\ = {x}^{′} {\vec{I}}^{′} + {y}^{′} {\vec{J}}^{′} = \\ = {x}^{′} ( cos α \cdot \vec{I} + sin α \cdot \vec{J} ) + y ( sin α \cdot \vec{I} + cos α \cdot \vec{J} ) = \\ = ( {x}^{′} cos α - {y}^{′} sin α ) \vec{I} + ( {x}^{′} sin α + y cos α ) \vec{J}\end{matrix}$$

These expressions define the “old” coordinates in the new reference system. By solving this equation system we obtain:

$$\begin{matrix}{x}^{′} = x cos α + y sin α \\ {y}^{′} = - x sin α + y cos α\end{matrix} \} \qquad ( 4 )$$

Expressions (3) and (4) are nothing else than the transformation formulas of the rotation of the axis. Therefore, it becomes possible to write the following relationship for the translation:

$$\{ \begin{matrix}{x}^{′} = x - a \\ {y}^{′} = y - b\end{matrix} \qquad ( 5 )$$

Translation Combined with Rotation

In the case of a translation combined with rotation, the equations are:

$$\{ \begin{matrix}{x}^{′} = ( x - {x}_{0} ) \cdot {M}_{X} \cdot cos α + ( y - {y}_{0} ) \cdot {M}_{y} \cdot sin α \\ {y}^{′} = ( x - {x}_{0} ) \cdot {M}_{X} \cdot sin α + ( y - {y}_{0} ) \cdot {M}_{y} \cdot cos α\end{matrix} \qquad ( 6 )$$

Where
Mₓ the X axis scale
M_{y} the scale on the Y axis
x₀ and y₀ are the coordinates of reference point or landmark.

These elementary operations, which are not presented in an exhaustive list here, applied to acquired images, help achieve what was stated above: 3D reconstruction, positioning and referencing images. The main advantage of an image or a 3D object based on the fact that you can return at will in any direction without having to go search for other images scattered on the disk.

Equations (6) would refocus the subsequent images on a 3D model after rotation and shift of the capsule along its trajectory.

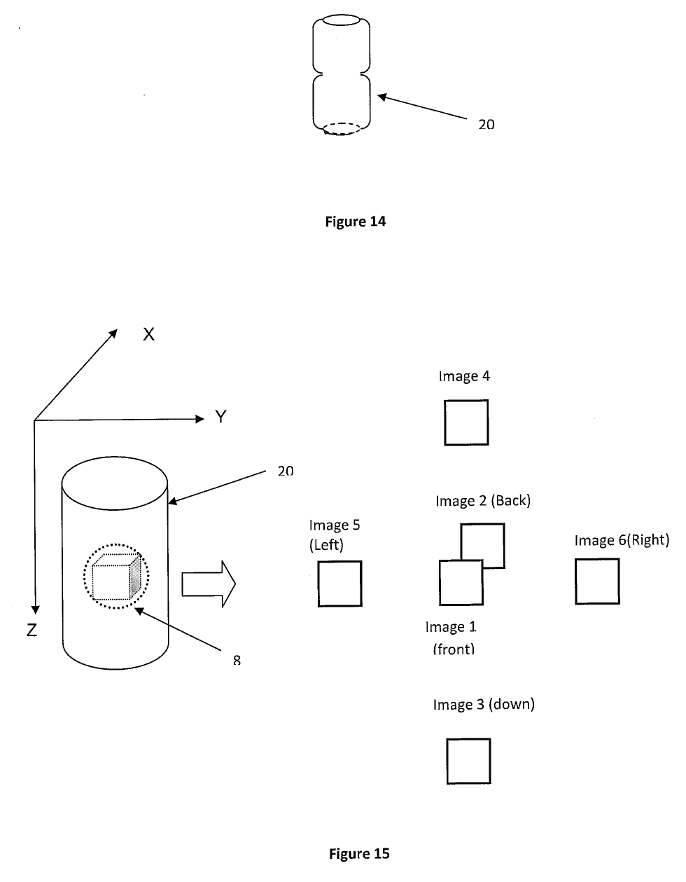

From the images taken by the system and using the principles exposed, it is possible to reconstruct 3D objects, for example the one illustrated in FIG. 14 which is a part of the digestive tract 20. It is to be noted that the images taken by the capsule correspond to the inner surface of the object being inspected.

Going back to the method described above, let's consider a capsule 8 containing a least six image sensors (as illustrated and described in reference to FIG. 1) This capsule is placed in a 3-axis reference system: X, Y, Z (FIGS. 9 and 10). The sensors are assumed to be mounted in parallel two by two, so that each of them can be parallel to a plan of the coordinates system (see figures.

Let's consider (x0, y0, z0), the initial point of image recording/sensing. This initial point can be defined in different ways. For example, when the capsule is on the patient tongue, before being swallowed, a radio frequency (RF) signal is transmitted from an external device to the capsule receiver.

As said above, a first set of images is captured by all sensors at that initial moment.

The image processing features can be implemented in an external equipment (belt and work station), but it can also be implemented partially or totally in the capsule. Anyway, the tasks distribution can be decided during the design and depending on the application.

For the first set of images, (x0,y0,z0), each sensor captures the full image. The full series of these images is sent to the receiver (or stored in a memory in the capsule for future treatment).

A signal may be sent to confirm that this is the initial capturing position.

Taking into account the image sizes and the bandwidth limitation, the throughput and the possible speed of the capsule motion, it might be difficult to transmit full images. In case of small pixel count sensors, the transmission of full images is possible. So, the processing is external performed by the workstation or other suitable treatment means.

This initial step is illustrated in FIG. 15 with full images from six sensors (Image 1, Image 2, Image 3, Image 4, Image 5 and Image 6).

In the case of higher definition sensors, or if a high number of sensors is used (thus increasing the data to be transmitted), some technique are introduced in the capsule devices for on-chip pre or processing in order to reduced the transmitted information.

That can be done by image compression for example.

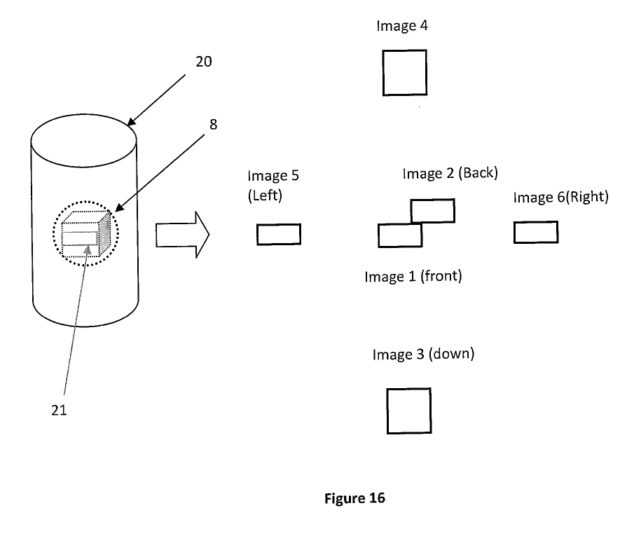

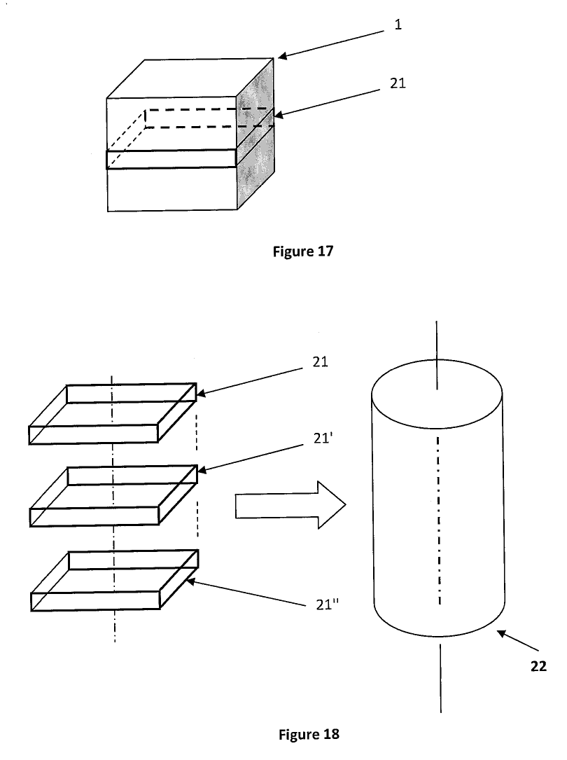

Another way to reduce the needed communication throughput is described hereafter whereby some images are reduced to slices as illustrated with reference to FIGS. 16 to 18.

From the starting point (x0, yo, z0), to the next image set, (x1, y1, z1), the image analysis allows the detection of the movement direction. That is to say, instead of transmitting to the belt or memory means a set of full images, it is enough to shoot pictures at a given frequency, high enough so that any image from any sensor contains a part of the previous image. This is just the principle of Shannon Theorema.

In FIG. 16, as illustrated, Image 3 and Image 4 are fully transmitted whereas only a slice 21 of Image 1, Image 2, Image 5 and Image 6 is transmitted.

In these conditions,
Only front and back images are fully transmitted.
To avoid bandwith saturation, a slice of other captured images are transmitted.

For example, if the capsule has been moved only in Y direction, without rotation, the slice of image 1 shown FIG. 4.
The slices are defined to be parallel to the movement direction of the capsule.

The size of slices 21 is not definitively fixed. One can implemented a fixed size, a variable one or an adaptative slice size. It becomes clear that there are two limits (upper and lower): full size (full image) and one line image. One line image means, at every clock period, only one line is transmitted. This is possible.

### Choosing the Slice Position in an Image.

Assuming a slice 21 of one line only.

This slice 21 is chosen for example in the middle of each picture as illustrated in FIGS. 16 (case of capsule shift along Y-axis) and 17.

At the beginning, the capsule trajectory is not known. In order to detect the movement, one does the following steps:

Two consecutive sets of full images are taken from all sensors. Because these two set are captured with a time difference, image processing techniques allow detection of rotation, shifting of the capsule. The internal clock of the capsule chips is used to define the frequency of image shooting.

Taking the first set of images as a reference, the second set is compared to extract movement: shift, rotation, etc. So, the same analysis allow to define coordinates (X1, Y1, Z1) of the second set.

At this second position of the capsule, knowing the actual direction of movement the capsule transmits:
2 full images (front and back)
A slice image from each other.

These slices 21 are selected around the axis movement, which can be in any direction of (X, Y, Z).

The capsule coordinates, detected as described, are also sent to the external receiver/memory to allow to build the 3D model of capsule tract.

The reconstruction of a 3D object 22 from slices-images 21, 21′, 21″ is illustrated with reference to FIG. 18.

Following the coordinated obtained from image analysis of the capsule movement and putting slices each after another, one creates a 3D-object like in computer aided tomography.

In the case of slice images transmission, full images are not always from the same sensors. In fact, even sending slices, the aim is to show always the front and back sides of the capsule. The front and back sides are defined according to the movement direction of the capsule. Depending on the capsule rotation, back/side can be images taken by any of all sensors. That is to say, the two full images can be from any image sensors, or mixed (case: one full image made of two parts/slices from two different sensors).

By the way, front and back images described here are no more else these shown by existing capsules equipped with two image sensors. However because of the oval shape of these capsules, in the case of rotation the front and back views are not guaranteed. The spherical capsule allows always front and back full view because of its spherical shape and 360° view.

### Adding Surface Shape or Relief in 3D-Object

Over the past ten years, there has been significant research on video endoscopy to create 3D reconstruction of the digestive tract. Scientists have approach this challenge through hardware and software improvements.

In the 3D object shown in FIG. 7, the drawback is the absence of relief, i.e. surface shape. In fact, because there is only one sensor for each direction, there is no relief in shape of the object.

Of course, some techniques exist in the literature allowing to obtain depth information.
Stereo endoscopy systems have been proposed to capture stereo-images and to create depth information and therefore 3D construction of digestive structures. However, due to size and compactness issues such systems have not been widely accepted.
Some Software approaches based on single images (monocular). Other are based on stereo techniques and geometric constraints from multiple frame to perform 3D reconstruction.
Other scientists use methods that fuse different modalities (MRI, CT) are utilized along with endoscopy to perform 3D reconstruction.

Many of the described techniques are in the literature and free of use. They can be applied in the described capsule to provide different versions of the device.

The shape of surface is one thing, the accuracy of dimensions is another one. In diagnosis with capsule endoscopy, doctors and surgeons need also to know the dimensions of the lesions. This is more useful especially when following the development of a disease and treatment evaluation.

In the past, stereovison and photogrametry have show efficiency.

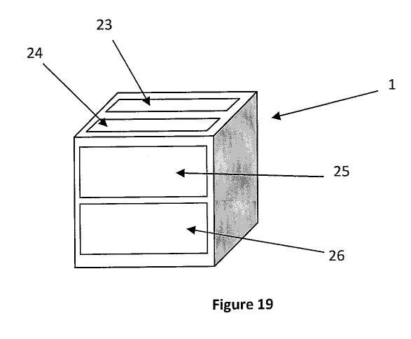

So, in order to provide more accurate depths and dimension, another embodiment of capsule is proposed. The main difference is about the image sensors: two image sensors are inserted to each face of the core base 1, instead of one single sensor. This embodiment is illustrated in FIG. 19 where the core base 1 comprises two image sensors (for example 23-24 and 25-25) on each face. In the illustration of FIG. 19, sensors are only illustrated on two faces but of course, it is intended to place two such sensors on each face of the core base in accordance with the principles of the present invention.

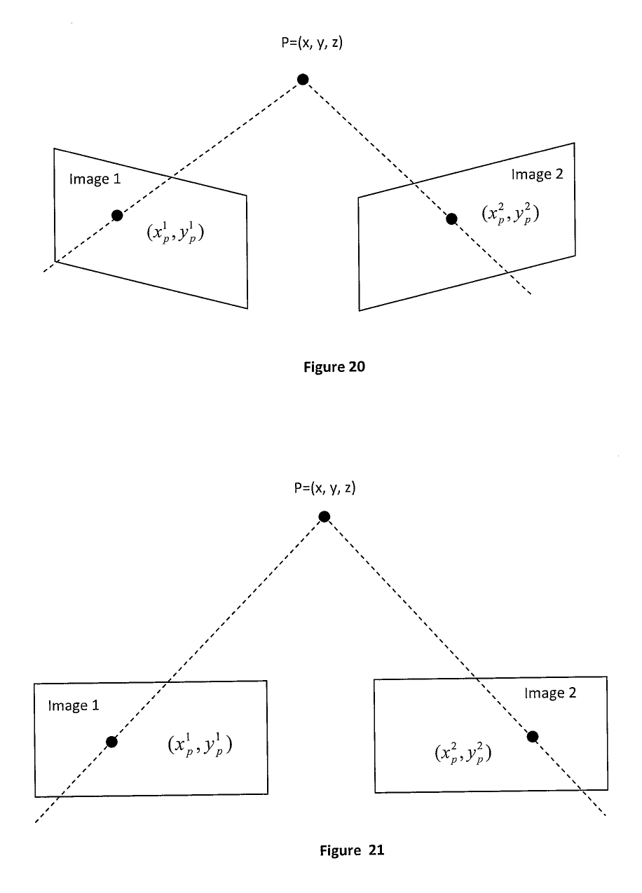

There are two possibilities in placing these sensors 23-26: on the same flat surface with the same angle, or with different angles this being illustrated in FIGS. 20 and 21. The same treatment as described above may be applied to the images and data provided by this configuration of sensors 23-26.

The capsule according to the invention can be powered by either a battery or by transponder power supplied via a remote antenna coupling. In the latter case, an external source transmits energy to the capsule via an antenna built into the capsule. The power transmission antenna has the advantage of providing an outlet for recharging the batteries included in a device carried in a belt for example. This has the effect of lengthening the time of registration.

The device and method according to the present invention thus allows a 3D reconstruction of a body part of the user, for example of the digestive tract, from views of the image sensors and provide real scale and relief.

### Image and Data Transmission

The transmission of images and data may rely on such a chip Bluetooth®. This would inherit a standardized and mastered technology. Such an approach would also allow Bluetooth® to better manage the power consumption of the capsule, using the low-power modes defined by the Bluetooth® standard. Moreover, because of the wide band Bluetooth® and its spectrum channel hopping, the transmission quality will be good. There is also a choice between three power classes of Bluetooth®. Outside of Bluetooth® communication standard can all be used.

Of course, all the examples and embodiments described herein are for illustration purposes and should not construed in a limiting manner as to the scope of the invention. Variations by way of equivalent means are possible.

### LIST OF TECHNICAL REFERENCES (ALL INCORPORATED BY REFERENCE)

O. Dewit, “Video-capsule endoscopy”, Louvain Medical 2008, 127, 1: 43-45.
G. Gay, I. Fassler, M. Delvaux, “states current recommendations for the use of video-capsule endoscopy in Europe,” ESGE Guidelines, Paris November 2003.
Delvaux et al, “Minimal Standard Terminology for Capsule Endoscopy” gastrointesti Surg 2003; 1857 (abstract).
Michel Coster, J L Cherm, “Accurate analysis of images, Presses du CNRS, 1989.
Gerard Gay, Michel Delvaux, Rene Laugier,” The double balloon enteroscopy (DBE), “Recommendation of the French Society of Digestive Endoscopy (SFED), March 2006.
G. Gay, M. Delvaux, I. Fassler, “The Double Balloon Enteroscopy: Principles, Methodology, results and indications,” Endoscopica, Volume 35—No. 3—2005, pages 317-327
Alexandre Karargyris, Orestis Karargyris, and Nikolaos Bourbakis; <<3D representation of the digestive tract surface in Wireless Capsule Endoscopy videos>>; 2010 IEEE Internationl Conference on Bioinformatics and Bioengineering. pages 279-280.
U. Mueller-Richter et al <<Possibilities and limitations of current stereo-endoscopy in Surgical endoscopy>>, Vol. 18, Number, pages 942-947, June 2004

1. An imaging device, for example a endoscopic capsule, comprising a core base having a plurality of faces, said faces supporting at least one an imaging device with an illumination means, said device further comprising electronic means and being contained in a spherical envelope.

2. The device according to claim 1 comprising at least six faces forming a cube.

3. The device according to claim 1, wherein said illumination device is placed around said imaging device.

4. The device of claim 1, wherein said illumination device is placed within said imaging device.

5. The device of claim 1, comprising means allowing an orientation of the device.

6. The device of claim 1, wherein said orientation means comprise inductors placed such that there is one pair of them in each axis-direction, i.e. in each plane: (X,Y), (X,Z), and (Y,Z).

7. The device as defined in claim 1 wherein it comprises at least two different imaging devices per face.

8. The device of claim 7, wherein the two imaging devices are aligned with one another or have an angle between them.

9. An apparatus comprising at least a device according to claim 1.

10. A reconstruction method using the device of claim 1 for building a 3D image of a user's body part from views of the image sensors.
